(19) United States
(12) Patent Application Publication
(10) Pub. No.: US 2024/0115476 A1
(43) Pub. Date: Apr. 11, 2024

(54) COMPOSITIONS COMPRISING A MULTI-TAIL SURFACTANT AND A CATIONIC POLYMER AND USES

(71) Applicant: RHODIA OPERATIONS, Aubervilliers (FR)

(72) Inventors: Chloe MOREAU, La Plaine Saint Denis (FR); Margot BON, Paris (FR); Anne-France LERON, Massy (FR); Martin VETHAMUTHU, Princeton, NJ (US)

(21) Appl. No.: 18/264,296

(22) PCT Filed: Feb. 8, 2022
(86) PCT No.: PCT/EP2022/052933

§ 371 Date: Aug. 4, 2023

(30) Foreign Application Priority Data
Feb. 8, 2021 (US) 63/146849

(51) Int. Cl. A61K 8/46; A61K 8/41; A61K 8/49; A61K 8/73; A61Q 5/00; A61Q 5/02

(52) U.S. Cl. CPC A61K 8/466; A61K 8/416; A61K 8/463; A61K 8/4926; A61K 8/737; A61Q 5/006; A61Q 5/02; A61K 2800/5426; A61K 2800/596

(57) ABSTRACT

The present invention provides compositions comprising an active ingredient, with a combination of at least one multi-tail surfactant, at least one cationic polymer and at least one anionic surfactant, different from the at least one multi-tail surfactant.

### FIELD

The present invention relates to the technical field of compositions comprising an active ingredient, in particular to personal care compositions or household care compositions.

More precisely, the invention provides compositions comprising an active ingredient, with a combination of at least one multi-tail surfactant, at least one cationic polymer and at least one anionic surfactant, different from the at least one multi-tail surfactant.

### BACKGROUND

An important challenge for a composition comprising an active ingredient is to maximise the quantity of the active ingredient which is delivered on its point of action. In particular, when the composition is delivered or deposited on a substrate, it is desirable to obtain a high deposition level of the active ingredient, by comparison to the quantity of the active ingredient present in the composition. Additionally, when a subsequent operation occurs after this delivery, a challenge is also to obtain the retention of the active ingredient, to maintain its action over time. Often, for personal care compositions or household care compositions, the delivery of the composition is carried out with a dilution step and/or a rinsing step that may alter the deposition and/or the retention of the active ingredient on the substrate where its benefit effect is needed.

A typical example of this challenge may be found in the hair care compositions, for instance in antidandruff shampoos which offer rinse-off treatment products with application ease, time-saving, and general convenience. As explained in US 2020/0129402, historical solutions proposed shampoo formulations that used cationic polymer with anionic surfactants to form coacervates. These formulations were paired with particulate agents, and the coacervate used to increase the deposition of the insoluble particulate anti-dandruff agents.

But problems were encountered with these historical proposals: coacervate leads to indiscriminate particle deposition to both the scalp and hair, which could cause undesirable hair aesthetics; the particulate anti-dandruff agents opacify the product, which leads to limitations in visual product aesthetics; the deposited particulate anti-dandruff agent is wasted.

It was proposed to bypass the limitations associated with particulate antidandruff agents by switching to anti-dandruff agents that are soluble in surfactant-based formulations. But in that case, the main issue is to achieve suitable deposition levels of the antidandruff agent on the surface of the hair and scalp, since most of it is rinsed-out with water-soluble micelles. To combat the deposition problem, again the approach can be to make the soluble antidandruff agent particulate (e.g. encapsulating the soluble antidandruff agent). While these approaches improve the spatial distribution and dissolution, the problems associated with particulate antidandruff agent deposition reappear.

The recent solution disclosed in US 2020/0129402 proposes a hair care composition comprising:
a) from about 8% to about 25% of one or more surfactants;
b) from about 0.01% to 10% of one or more surfactant-soluble agent;
c) with a specific condition using a given fractional soluble agent concentration (a).

The present invention is directed to solve the above mentioned problems. The present invention proposes compositions which are suitable for any kind of active ingredient. In particular, it appears that there is a genuine need for improving the way the compositions containing an active ingredient deliver and maintain this active ingredient on its point of action. In this context, the main aim of this invention is to provide compositions which allow the action of an optimal amount of the active ingredient.

### SUMMARY OF THE INVENTION

According to a first aspect, the invention concerns a composition comprising:
a multi-tail surfactant A,
a cationic polymer B,
an active ingredient C,
an anionic surfactant D which is different from the multi-tail surfactant A,
wherein, in the composition, the weight amount of the multi-tail surfactant A is lower than the weight amount of the anionic surfactant D.

The invention allows improving the action of the active ingredient C, by the combined use of the multi-tail surfactant A and of the cationic polymer B. Improving the action of the active ingredient C means that the major amount of the active ingredient C included in the composition can have the action for which it is included in the composition. The invention is particularly suited for personal care compositions or household care compositions. When, these compositions are intended to be deposited on a substrate (typically, skin, hair, scalp, textiles), the active ingredient C will be better retained on the targeted substrate with the compositions of the invention, even after addition of water or after a rinsing step with water, as classically carried out with hair care compositions, like shampoo.

The inventors surprisingly demonstrated that the multi-tail surfactant A and the cationic polymer B have a synergistic effect when the composition of the invention is used. Without limiting the present invention to any one theory or mode of action, hydrophobic structures made possible with the claimed composition, lead to a higher amount of the active ingredient C trapped in such structures and therefore deposited on the substrate upon dilution during rinsing. In other words, the multi-tail surfactant A and the cationic polymer B act together to drastically increase the deposition, and most of all, the retention of the active ingredient C, on the substrate where its action is desirable, in particular the hair and scalp for shampoo, as antidandruff shampoo. The presence of the anionic surfactants D, also, contributes to this improvement.

Advantageously, the composition of the invention is a personal care composition or a household care composition, in particular a hair care composition, especially a shampoo.

In general, the multi-tail surfactant A used in the invention has at least two linear or branched alkyl chain, with at least one of these chains, preferably each of these chains, having at least 6 carbon atoms.

The compositions in accordance with the invention preferably exhibit one or more of the following features, or any combination of these features, or even all of the features below when they are not mutually exclusive:

The multi-tail surfactant A may represent at most 10% by weight and/or at least 0.1% by weight, advantageously from 0.1 to 10% by weight, and preferably from 0.1 to 5% by weight of the total weight of the composition.

According to preferred embodiments, the multi-tail surfactant A is a dialkylsulfosuccinate, which represents less than 10% by weight and/or at least 0.5% by weight, preferentially from 1% to 7% by weight, and more preferentially from 1 to 5% by weight, of the total weight of the composition.

According to more preferred embodiments, the multi-tail surfactant A is dioctyl sodium sulfosuccinate, sodium bis(tridecyl)sulfosuccinate or a mixture thereof, which represent less than 10% by weight and/or at least 0.5% by weight, preferentially from 1 to 7% by weight, and more preferentially from 2 to 5% by weight, relative to the total weight of the composition.

The cationic polymer B may be a cationic guar polymer, especially selected among guar hydroxypropyltrimonium chloride and hydroxypropyl guar hydroxypropyltrimonium chloride.

According to preferred embodiments, the composition includes at least 0.01% by weight relative to the total weight of the composition, advantageously from 0.1 to 5% by weight, and preferably from 0.1 to 1% by weight, of the cationic polymer B, and in particular of a cationic guar polymer.

According to more preferred embodiments, the composition includes at least 0.01% by weight relative to the total weight of the composition, advantageously from 0.1 to 5% by weight, and preferably from 0.1 to 1% by weight, of guar hydroxypropyltrimonium chloride, hydroxypropyl guar hydroxypropyltrimonium chloride or a mixture thereof.

A composition of the invention may include at least 0.01% by weight, advantageously from 0.05 to 10% by weight, and preferably from 0.1 to 5% by weight, of the active ingredient C, relative to the total weight of the composition.

According to preferred embodiments, the composition includes from 0.05 to 10% by weight, relative to the total weight of the composition, and preferably from 0.1 to 5% by weight of a hydrophobic active ingredient C, and in particular of an anti-microbial or an anti-fungal agent.

According to more preferred embodiments, the active ingredient C is piroctone olamine, which represents advantageously from 0.05 to 5% by weight, and preferably from 0.1 to 1% by weight of the total weight of the composition.

The composition of the invention may include at least 5% by weight, advantageously from 5 to 20% by weight, and preferably from 5 to 15% by weight of the anionic surfactant D, relative to the total weight of the composition.

According to preferred embodiments, the anionic surfactant D is a sulfated anionic surfactant, and preferably is sodium laureth sulfate.

According to more preferred embodiments, the anionic surfactant D is sodium laureth sulfate which represents from 5 to 20% by weight, and preferably from 8 to 15% by weight of the total weight of the composition.

The composition of the invention may also include one or several additional surfactant(s) selected among amphoteric, zwitterionic and non-ionic surfactants.

According to preferred embodiments, the composition includes an additional surfactant, in particular selected among sulfate-free single tail surfactants, such as sodium lauroyl sarcosinate, sodium lauroamphoacetate, cocamidopropyl betain, and decyl glucoside.

According to more preferred embodiments, the composition includes an amphoteric surfactant, which is present in the composition in an amount from 0.01 to 20% by weight, and preferably from 0.1 to 10% by weight, relative to the total weight of the composition.

Advantageously, the amphoteric surfactant which is present in the composition is cocamidopropyl betain and represents from 0.01 to 20% by weight, and preferably from 0.1 to 10% by weight, of the total weight of the composition.

Such above-cited mentioned preferred ranges and components are particularly suitable for obtaining an optimal improvement of the deposition and/or retention of the active ingredient C.

The composition according to the invention may also comprise an oil, and in particular a non-toxic or cosmetic oil, in an amount of 10% by weight or less, relative to the total weight of the composition.

According to preferred embodiments, the composition according to the invention is an aqueous composition, which preferably comprises from 40 to 85% by weight of water, relative to the total weight of the composition.

The composition of the invention may also comprise a solvent selected among lower alkyl alcohols and/or polyhydric alcohols, in particular selected among ethanol, isopropanol, propylene glycol, hexylene glycol, glycerin, and propane diol. The invention also provides the use of the combination multi-tail surfactant A/cationic polymer B in a composition according to the invention, for improving the deposition and/or retention of the active ingredient C, when the composition is applied on a targeted substrate where its action is needed. Advantageously, the composition is a shampoo, in particular an antidandruff shampoo, and the targeted substrate is hair and/or scalp.

In particular, the combination multi-tail surfactant A/cationic polymer B in a composition according to the invention, is used to improve the retention of the active ingredient C, after a rinsing step with water.

### Definitions

All documents cited in the present specification are hereby incorporated by reference in their entirety.

Unless otherwise specified, all terms used in the disclosure of the invention, including technical and scientific terms, have the meaning as commonly understood by one of ordinary skill in the art to which this invention belongs. By means of further guidance, some term definitions are included to better understanding.

Throughout this specification, unless the context requires otherwise, the word “comprise”, or variations such as “comprises”, “comprised” or “comprising”, ae synonymous with “include” or “contain”, and the corresponding variations. So, these terms will be understood to imply the inclusion of a stated element or method step or group of elements or method steps, but not the exclusion of any other non-cited element or method step. The word “comprise”, “include” or “contain” encompasses “consist essentially of” and “consist exclusively of”. The compositions of the present invention can comprise, consist essentially of, or consist exclusively of, the essential components as well as optional ingredients described herein. As used herein, “consisting essentially of” means that the composition or component may include additional ingredients, but only if the additional ingredients do not materially alter the basic and novel characteristics of the compositions, uses or methods of the invention.

As used in the subject specification, the singular forms “a”, “an” and “the” include one of the designated entity or several of the designated entity, unless the context clearly dictates otherwise (i.e. the designated entity can only be one entity, for instance sodium laureth sulfate). The articles “a” and “an” when used in a claim, are understood to mean one or more of what is claimed or described. Thus, for example, reference to “a multi-tail surfactant A”, “a cationic polymer B”, “an active ingredient C”, or “an anionic surfactant D” include a single multi-tail surfactant A, cationic polymer B, active ingredient C, or anionic surfactant D, respectively, as well as at least one, and in particular two or more of the specified component. So, for instance, “a multi-tail surfactant A” encompasses a mixture of different components covered by the definition “a multi-tail surfactant A”. Reference to “the invention” includes single or multiple aspects taught by the present disclosure.

Where amount ranges are given, these are to be understood as being the total amount of said ingredient in the composition, or where more than one species fall within the scope of the ingredient definition, the total amount of all ingredients fitting that definition, in the composition. For instance, when it is specified that the composition comprises from 0.1% to 1% by weight of the active ingredient C, if the active ingredient C corresponds to a mixture of two active ingredients, the total amount of this mixture represents from 0.1% to 1% by weight of the total amount of the composition.

All given percentages are weight % based upon the total weight of the composition of the present invention, unless otherwise specified. All such weights as they pertain to listed ingredients are based on the active level and, therefore, do not include carriers or by-products that may be included in commercially available materials.

The total amount of the different components of the composition described hereinafter can account for up to 100% (or 100%) of the total weight of the compositions of the present invention.

The recitation of numerical ranges by endpoints includes all numbers and fractions subsumed within the respective ranges, as well as the recited endpoints. All numeric ranges are inclusive of narrower ranges; delineated upper and lower range limits are combinable to create further ranges not explicitly delineated.

The term “about” as used herein when referring to a measurable value such as a parameter, an amount, a temporal duration, and the like, is meant to encompass variations of and from the specified value, in particular variations of +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated.

As used herein, “molecular weight” refers to the weight average molecular weight unless otherwise stated. Molecular weight is measured using industry standard method, gel permeation chromatography (“GPC”).

“Charge density” refers to the term “charge density” refers to the ratio of positive charges on a monomeric unit of which a polymer is comprised to the molecular weight of said monomeric unit. The charge density multiplied by the polymer molecular weight determines the number of positively charged sites on a given polymer chain. It may be measured using industry standard method, such as Proton Nuclear Magnetic Resonance spectroscopy (“¹H NMR).

### DETAILED DESCRIPTION

Multi-Tail Surfactant (A)

Any multi-tail surfactants A can be used according to the invention, especially those which may be used in a personal care composition or in a household care composition. Such multi-tail surfactants A are well known in the art, and for instance described in U.S. Pat. No. 10,058,498. The relevant part of U.S. Pat. No. 10,058,498 is included hereafter.

Multi-tail surfactants A are surfactants which comprise at least one, preferably more than one, hydrocarbon (typically alkyl) chain including at least 6 carbon atoms. Multi-tail surfactants include anionic, cationic, zwitterionic, amphoteric surfactants, and combinations thereof, having more than one hydrocarbon (typically alkyl) chain. The at least two hydrocarbon chains can be aromatic or aliphatic, straight or branched hydrocarbon chains, typically alkyl chains, and can have one or more moieties on the hydrocarbon chains comprising a solvophobic group (i.e., lacking an affinity for a specific solvent, for example, water) and/or a solvophilic group (i.e., having an affinity for a specific non-polar or low polar solvent). More specifically, but not by way of limitation, the hydrocarbon chains of the multi-tail surfactants A are preferably hydrophobic in the presently disclosed and/or claimed inventive concept(s) so as to form more stable and denser hydrophobic structures on the active ingredient. Examples of multi-tail surfactants A include, but are not limited to, dioctyl sulfosuccinates like sodium dioctyl sulfosuccinate, and quaternary ammonium compounds with long alkyl chains like dicoco dimethylammonium chloride, dipalmitoylethyl hydroxyethylmonium methosulfate, and dialkyl ammonium methosulfate. Multi-tail surfactants such as those marketed under the trade names STEPANTEX® DC 90 (Stepan Company, Northfield, Ill.), STEPANQUAT® GA-90 (Stepan Company, Northfield, Ill.), ARQUAT® 2C-75 (AkzoNobel, Chicago, Ill.) and AEROSOL® OT (Cytec Industries Inc., West Paterson, N.J.) are also useful in the present invention. Often, these surfactants A are available, as formulations in a solvent, such as lower alkyl alcohols and polyhydric alcohols, typically propylene glycol or hexylene glycol. These formulations may be used for the preparation of the compositions according to the invention.

The alkyl chains of the multi-tail surfactants A can be the same or different. Preferentially, the alkyl chains include 6 to 20 carbon atoms, have preferably at least 8 carbon atoms, more preferably 8 to 15 carbon atoms.

A preferred class of multi-tail surfactants A corresponds to dialkylsulfosuccinate which are anionic surfactants. Preferentially, their alkyl chains include 6 to 20 carbon atoms, have preferably at least 8 carbon atoms, more preferably 8 to 15 carbon atoms. In general, they would be used as their sodium or ammonium salt. According to the invention, the sodium dioctyl sulfosuccinate and the sodium bis(tridecyl)sulfosuccinate are preferred multi-tail surfactants.

Advantageously, the multi-tail surfactant A is present in the composition in an amount of at most 10% by weight and/or at least 0.1% by weight, preferably from 0.1 to 10% by weight, and preferentially from 0.1 to 5% by weight.

In particular, the compositions according to the invention include less than 10% by weight and/or at least 0.5% by weight, advantageously from 1% to 7% by weight, and preferably from 1 to 5% by weight of a dialkylsulfosuccinate, and in particular of a dialkylsulfosuccinate previously described, and more preferably less than 10% by weight and/or at least 0.5% by weight, preferentially from 1 to 7% by weight, and preferably from 2 to 5% by weight of dioctyl sodium sulfosuccinate, sodium bis(tridecyl)sulfosuccinate or a mixture thereof.

According to preferred embodiments, the compositions according to the invention include dioctyl sodium sulfosuccinate, sodium bis(tridecyl)sulfosuccinate or a mixture thereof, as multi-tail surfactant A, and advantageously only dioctyl sodium sulfosuccinate, sodium bis(tridecyl)sulfosuccinate or a mixture thereof, as multi-tail surfactant A.

Cationic Polymer (B)

The compositions of the invention comprise a cationic polymer B. Any cationic polymers B can be used according to the invention, especially those which may be used in a personal care composition or in a household care composition. Such cationic polymers B are well known in the art, and for instance described in US 2020/0129402. The relevant disclosure of US 2020/0129402 is included hereafter.

Suitable cationic polymer B includes (a) cationic guar polymers, (b) cationic non-guar galactomannan polymers, (c) cationically modified starch polymers, and in particular cationic tapioca polymers, (d) cationic copolymers of acrylamide monomers and cationic monomers, (e) synthetic, non-crosslinked, cationic polymers, which may or may not form lyotropic liquid crystals upon combination with the surfactant (f) cationic cellulose polymers.

Cationic Guar Polymers (a)

The compositions may comprise a cationic guar polymer, which is a cationically substituted galactomannan (guar) gum derivatives.

It may be for example a galactomannan that has been modified by chemical means, e.g. quaternization, with one or more derivatizing agents containing reactive groups.

The cationic guar polymer may be obtained for instance by reaction between the hydroxyl groups of the galactomannan and the reactive functional groups of the derivatizing agents.

Methods for the preparation of a cationic guar polymer are disclosed in U.S. Pat. Nos. 4,663,159; 5,473,059; 5,387,675; 3,472,840; 4,031,307; 4,959,464 and US 2010/0029929, all of which are incorporated herein by reference.

The cationic guar polymer of the invention contains at least one cationic group.

As used herein, the term “cationic” covers not only positively charged groups, but also groups which may become positively charged depending on the pH.

A cationic guar polymer of the invention is a guar polymer that has been chemically modified to provide said guar polymer with a net permanent positive charge in a pH neutral aqueous medium. Those that are non permanently charged, e.g. guar polymers that can be cationic below a given pH and neutral above that pH also fall within the scope of the present invention.

According to anyone of the invention embodiments, the terms “cationizing agents”, “cationic groups” and “cationic moieties” include ammoniums (which have a positive charge) but also primary, secondary and tertiary amines and their precursors (which can lead to positively charged compounds).

According to the invention, the guar polymer is derivatized or modified so as to contain a cationic group. The resulting compound is the guar derivative.

According to one of the invention embodiments, the guar derivatives of the invention result from the reaction of a guar, with a cationizing agent.

Cationizing agents of the present invention are defined as compounds which, by reaction with the hydroxyl groups of the guar can lead to a guar derivative comprising at least one cationic group according to the invention.

Cationizing agents of the present invention are defined as compounds which contain at least one cationic moiety. Cationizing agents comprise agents which can lead to cationic guar.

A group of suitable derivatizing reagents typically contain a reactive functional group, such as an epoxy group, a halide group, an ester group, an anhydride group or an ethylenically unsaturated group, and at least one cationic moiety or a precursor of such cationic moiety.

As used herein, the term “derivatizing agent” means an agent containing at least a cationic moiety which is grafted to a guar polymer. The term “derivatizing agent” encompasses the terms “cationizing agent” and “grafting agent”.

In one embodiment of the invention, the cationic moieties may be linked to the reactive functional group of the derivatizing agent by a bivalent linking group, such as an alkylene or oxyalkylene group. Suitable cationic moieties include primary, secondary, or tertiary amino groups or quaternary ammonium, sulfonium, or phosphinium groups.

The derivatizing agent can comprise a cationic moiety, or a precursor of a cationic moiety, that contains a cationic nitrogen moiety, more typically, a quaternary ammonium moiety. Typical quaternary ammonium moieties are trialkylammonium moieties, such as trimethylammonium moieties, triethylammonium moieties, or tributylammonium moieties, aryldialkylammonium moieties, such as benzyldimethylammonium moieties, and ammonium moieties in which the nitrogen atom is a member of a ring structure, such as pyridinium moieties and imidazoline moieties, each in combination with a counterion, typically a chloride, bromide, or iodide counterion.

According to one of the invention embodiments, examples of cationizing agents, which lead to cationic guar derivatives of the invention are:
cationic epoxides, such as 2,3-epoxypropyltrimethylammonium chloride, 2,3-epoxypropyltrimethylammonium bromide, 2,3-epoxypropyltrimethylammonium iodide;
chlorohydrin-functional cationic nitrogen compounds, such as 3-halogeno-2-hydroxypropyl trimethylammonium chloride, for example 3-chloro-2-hydroxypropyl trimethylammonium chloride,
cationic ethylenically unsaturated monomers or their precursors, such as trimethylammoniumpropyl methacrylamide chloride salt, trimethylammoniumpropyl methacrylamide methyl sulfate salt, diallyl dimethyl ammonium chloride, vinyl benzyl trimethylammonium chloride, dimethylaminopropyl methacrylamide (tertiary amine) precursors of cationic monomers, such as N-vinyl formamide, N-vinylacetamide (whose units can be hydrolyzed after polymerization or grafted onto vinyl amine units).

In one embodiment of the invention, the cationizing agents, which lead to cationic guar derivatives of the invention are cationic epoxides, such as 2,3-epoxypropyltrimethylammonium chloride, 2,3-epoxypropyltrimethylammonium bromide and 2,3-epoxypropyltrimethylammonium iodide.

According to the invention, the cationic groups may be introduced into a guar polymer by reacting the guar polymer starting material with a derivatizing agent which comprises a reactive functional group and at least one cationic moiety (or a precursor of cationic moiety).

According to the invention, the cationic groups present in the guar derivative are incorporated into the guar polymer starting material by reaction of the hydroxyl groups of said guar polymer with a cationizing agent.

Preferred cationic groups are chosen from the group consisting of: primary, secondary or tertiary amino groups, quaternary ammonium, sulfonium or phosphinium groups, and mixtures thereof. In a particular preferred embodiment, the cationic group is chosen from trialkylammonium groups, such as trimethylammonium groups, triethylammonium groups, tributylammonium groups, aryldialkylammonium groups, such as benzyldimethylammonium groups, and ammonium groups in which the nitrogen atom is a member of a ring structure, such as pyridinium groups and imidazoline groups, each in combination with a counterion, typically a chloride, bromide, or iodide counterion. Preferably, each cationic group contains at least one cationic charge.

The cationicity of the guar derivative can be expressed in terms of degree of substitution.

As used herein, the expression “cationic degree of substitution” (DScat) means the average number of moles of cationic groups per mole of sugar unit. The (DScat) may be measured by means of 1H-NMR (solvent: D2O).

Once the 1H NMR spectrum is obtained, the integration of the multiplet of peaks corresponding to the anomeric proton on all guar units, usually between 3.2-4.3 ppm, is normalized to unity. The peak of interest, the one corresponding to the methyl protons of the quaternary ammonium group on guar units, is centered around 1.8 ppm. This peak is integrated for 9 protons given that there are 3 methyl groups on the ammonium function. Therefore the calculation of the (DScationic) for the case of the cationizing agent 2,3-epoxypropyltrimethylammonium chloride is as follows:

$$DS = \frac{INTEGRAL\_N ( Me ) 3}{INTEGRAL\_anomeric \_proton} / 9$$

According to anyone of the invention embodiments, the guar derivative of the invention has a cationic degree of substitution (DScat) higher than or equal to about 0.08, for instance higher than or equal to about 0.09, for instance higher than or equal to about 0.10.

According to anyone of the invention embodiments, the guar derivative of the invention has a cationic degree of substitution (DScat) lower than or equal to about 0.30, for instance lower than or equal to about 0.25, for instance lower than or equal to about 0.20.

According to one of the invention embodiments, the guar derivative of the invention has a cationic degree of substitution (DScat) comprised between about 0.08 and about 0.30, for instance between about 0.09 and about 0.25, for instance between about 0.10 and about 0.25.

The cationicity of the guar derivative of the invention may also be expressed in terms of charge density. The cationic degree of substitution may be converted to a charge density through several methods.

The preferred method for calculating charge density of cationic guar derivatives uses a method that specifically quantifies the equivalents of quaternary ammonium groups on said guar.

For cationic guars obtained by reacting a guar gum with 3-chloro-2-hydroxypropyltrimethylammonium chloride or 2,3-epoxypropyltrimethylammonium chloride, the cationic charge density may be calculated from the cationic degree of substitution using the following equation:

Cationic charge density in mequivalents per gram (meq/g)=

$$\frac{{DS}_{cat}}{162 + 151 \times {DS}_{cat}} \times 1000$$

In general, the equation above depends on the group which is grafted to the guar.

As used herein, the term “charge density” refers to the ratio of positive charges on a monomeric unit of which a polymer is comprised to the molecular weight of said monomeric unit. The charge density multiplied by the polymer molecular weight determines the number of positively charged sites on a given polymer chain.

According to the present invention, the guar derivative has a charge density below about 1.2 meq/g, for instance from about 0.5 to about 1.2 meq/g. According to anyone of the invention embodiments, the guar derivative of the invention may further contain at least one hydroxyalkyl group.

According to the invention, the degree of hydroxyalkylation (molar substitution or MS) of the guar derivative of the invention means the number of alkylene oxide molecules consumed by the number of free hydroxyl functions present on the guar.

According to anyone of the invention embodiments, the guar derivative of the invention may have a degree of hydroxyalkylation (MS) comprised between about 0 and about 1.5, for instance between 0.1 and about 1.0.

According to one of the invention embodiments, the hydroxyalkyl group is a C1-C6 hydroxyalkyl groups, for instance chosen from the group consisting of: a hydroxymethyl group, a hydroxyethyl group, a hydroxypropyl group and a hydroxybutyl group.

A guar derivative of the invention containing at least one hydroxyalkyl group may be prepared for example by reacting the corresponding alkene oxides (such as for example propylene oxides) with the guar so as to obtain a guar derivative which has been modified with hydroxyalkyl group (for example hydroxypropyl groups).

By the expression “average molecular weight” of the guar derivative of the invention, it is meant the weight average molecular mass of said guar derivative.

The average molecular weight of a guar derivative may be measured by SEC-MALS (Size Exclusion Chromatography with detection by Multi-Angle Light-Scattering detection). A value of 0.140 for do/dc is used for the molecular weight measurements. A Wyatt MALS detector is calibrated using a 22.5 KDa polyethylene glycol standard. All calculations of the molecular weight distributions are performed using Wyatt's ASTRA software. The samples are prepared as 0.05% solutions in the mobile phase (100 mM Na2NO3, 200 ppm NaN3, 20 ppm pDADMAC) and filtered through 0.45 μm PVDF filters before analysis. The average molecular weights are expressed by weight.

According to anyone of the invention embodiments, the average molecular weight of the guar derivative of the invention is higher than about 1,000,000 g/mol, for instance higher than about 1,500,000 g/mol, for instance higher than about 2,000,000 g/mol.

According to anyone of the invention embodiments, the average molecular weight of the guar derivative of the invention is lower than about 3,500,000 g/mol, for instance lower than about 3,000,000 g/mol.

According to one of the invention embodiments, the average molecular weight of the guar derivative of the invention is comprised between about 1,000,000 g/mol and about 3,500,000 g/mol, for instance between about 1,500,000 g/mol and about 3,500,000 g/mol, for instance between about 2,000,000 g/mol and 3,000,000 g/mol.

Cationic Non-Guar Galactomannan Polymers (b)

The compositions of the present invention may comprise a galactomannan polymer derivative having a mannose to galactose ratio of greater than 2:1 on a monomer to monomer basis. The galactomannan polymer derivative may be selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and also is affected by climate. Non Guar Galactomannan polymer derivatives preferably used according to the invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include, but are not limited, to Tara gum (3 parts mannose/1 part galactose), Locust bean or Carob (4 parts mannose/1 part galactose), and Cassia gum (5 parts mannose/1 part galactose).

The non-guar galactomannan polymer derivatives may have a molecular weight from about 1,000 to about 10,000,000 g/mol, and/or from about 5,000 to about 3,000,000 g/mol.

The compositions of the invention can also include galactomannan polymer derivatives which have a cationic charge density from about 0.5 meq/g to about 7 meq/g. The galactomannan polymer derivatives may have a cationic charge density from about 1 meq/g to about 5 meq/g. The degree of substitution of the cationic groups onto the galactomannan structure should be sufficient to provide the requisite cationic charge density.

The galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include of the general formulas 1-5, as defined above.

Cationic non-guar galactomannan polymer derivatives formed from the reagents described above are represented by the general formula 6:

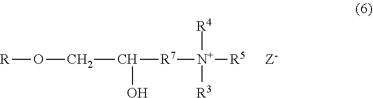

wherein R is the gum and R³, R⁴, R⁵ and R⁷ are as defined above. The cationic galactomannan derivative can be a gum hydroxypropyltrimethylammonium chloride, which can be more specifically represented by the general formula 7:

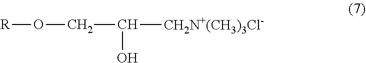

Alternatively the galactomannan polymer derivative can be an amphoteric galactomannan polymer derivative having a net positive charge, obtained when the cationic galactomannan polymer derivative further comprises an anionic group.

The cationic non-guar galactomannan can have a ratio of mannose to galactose greater than about 4:1, a molecular weight of about 1,000 g/mol to about 10,000,000 g/mol, and/or from about 50,000 g/mol to about 1,000,000 g/mol, and/or from about 100,000 g/mol to about 900,000 g/mol, and/or from about 150,000 g/mol to about 400,000 g/mol and a cationic charge density from about 1 meq/g to about 5 meq/g, and/or from 2 meq/g to about 4 meq/g and can be derived from a cassia plant.

Cationically Modified Starch Polymers, and in Particular Cationic Tapioca Polymers (c)

The compositions can comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cationically modified starch polymers disclosed herein have a percent of bound nitrogen of from about 0.5% to about 4%.

The cationically modified starch polymers for use in the composition can have a molecular weight about 850,000 g/mol to about 1,500,000 g/mol and/or from about 900,000 g/mol to about 1,500,000 g/mol.

The compositions can include cationically modified starch polymers which have a charge density of from about 0.2 meq/g to about 5 meq/g, and/or from about 0.2 meq/g to about 2 meq/g. The chemical modification to obtain such a charge density includes, but is not limited to, the addition of amino and/or ammonium groups into the starch molecules. Non-limiting examples of these ammonium groups may include substituents such as hydroxypropyl triammonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers generally have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“.sup.1H NMR”) methods well known in the art. Suitable.sup.1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be chosen from a variety of sources such as tubers, legumes, cereal, and grains. Non-limiting examples of this source starch may include corn starch, wheat starch, rice starch, waxy corn starch, oat starch, cassava starch, waxy barley, waxy rice starch, glutenous rice starch, sweet rice starch, amioca, potato starch, tapioca starch, oat starch, sago starch, sweet rice, or mixtures thereof.

The cationically modified starch polymers can be selected from degraded cationic maize starch, cationic tapioca, cationic potato starch, and mixtures thereof. Alternatively, the cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

An optimal form of the starch is one which is readily soluble in water and forms a substantially clear (% Transmittance of about 80 at 600 nm) solution in water. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm has been shown to be adequate for characterizing the degree of clarity of cosmetic compositions.

Suitable cationically modified starch for use in the compositions of the invention are available from known starch suppliers. Suitable cationically modified starch are nonionic modified starch that can be further derivatized to a cationically modified starch as is known in the art. Other suitable modified starch starting materials may be quaternized, as is known in the art, to produce the cationically modified starch polymer suitable for use in the compositions of the invention.

The degradation procedure of a starch may be carried out as follows: a starch slurry can be prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

Cationic Copolymers of Acrylamide Monomers and Cationic Monomers (d)

The compositions can comprise a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

The cationic copolymer can comprise:
(i) an acrylamide monomer of general formula AM:

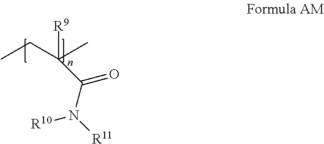

where R⁹ is H or a C1 to C4 alkyl; and R¹⁰ and R¹¹ are independently selected from H, C1 to C4 alkyl, —CH₂OCH3, —CH₂OCH₂CH(CH₃)₂, and phenyl, or together are a C3 to C6 cycloalkyl; and
(ii) a cationic monomer general formula CM:

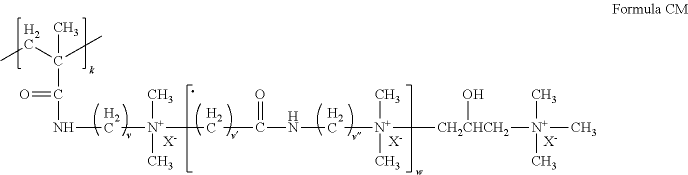

where k=1, each of v, v′, and v″ is independently an integer of from 1 to 6, w is zero or an integer of from 1 to 10, and X⁻ is an anion.

The cationic monomer can have the general formula CM and where k=1, v=3

and w=0, z=1 and X⁻ is Cl⁻, corresponds to the following structure:

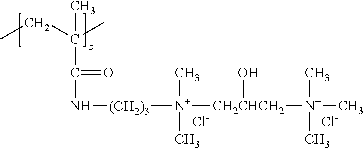

The above structure may be referred to as diquat. Alternatively, the cationic monomer can have the general formula CM and wherein v and v″ are each 3, v′=1, w=1, y=1 and X— is Cl⁻, corresponds to the following structure:

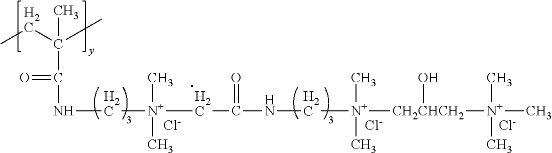

The above structure may be referred to as triquat.

Suitable acrylamide monomers include, but are not limited to, either acrylamide or methacrylamide.

The cationic copolymer (d) can be AM:TRIQUAT which is a copolymer of acrylamide and 1,3-propanediaminium,N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride. AM:TRIQUAT is also known as polyquaternium 76 (PQ76). AM:TRIQUAT may have a charge density of 1.6 meq/g and a molecular weight of 1.1 million g/mol.

Further, the cationic copolymer (d) may be of an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer (d) can comprise a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer (d) can be water-soluble. The cationic copolymer may be formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. The cationized esters of the (meth)acrylic acid containing a quaternized N atom may be quaternized dialkylaminoalkyl (meth)acrylates with C1 to C3 in the alkyl and alkylene groups. Suitable cationized esters of the (meth)acrylic acid containing a quaternized N atom can be selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom may be dimethylaminoethyl acrylate, which is quaternized with an alkyl halide, or with methyl chloride or benzyl chloride or dimethyl sulfate (ADAME-Quat). The cationic monomer when based on (meth)acrylamides can be quaternized dialkylaminoalkyl(meth)acrylamides with C1 to C3 in the alkyl and alkylene groups, or dimethylaminopropylacrylamide, which is quaternized with an alkyl halide, or methyl chloride or benzyl chloride or dimethyl sulfate.

Suitable cationic monomer based on a (meth)acrylamide include quaternized dialkylaminoalkyl(meth)acrylamide with C1 to C3 in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl(meth)acrylamide, all monomers that can be regarded as stable to the OECD hydrolysis test. The cationic monomer can be hydrolysis-stable and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

The cationic copolymer can have a molecular weight from about 100 thousand g/mol to about 1.5 million g/mol, or from about 300 thousand g/mol to about 1.5 million g/mol, or from about 500 thousand g/mol to about 1.5 million g/mol, or from about 700 thousand g/mol to about 1.0 million g/mol, or from about 900 thousand g/mol to about 1.2 million g/mol.

The cationic copolymer (d) can be a trimethylammoniopropylmethacrylamide chloride-N-Acrylamide copolymer, which is also known as AM:MAPTAC. AM:MAPTAC may have a charge density of about 1.3 meq/g and a molecular weight of about 1.1 million g/mol. The cationic copolymer can be AM:ATPAC. AM:ATPAC can have a charge density of about 1.8 meq/g and a molecular weight of about 1.1 million g/mol.

Synthetic, Non-Crosslinked, Cationic Polymers (e)

The compositions can comprise a cationic synthetic polymer that may be formed from
i). one or more cationic monomer units, and optionally
ii). one or more monomer units bearing a negative charge, and/or
iii). a nonionic monomer,
   wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers.

The cationic polymers can be water soluble or dispersible, non-crosslinked, and synthetic cationic polymers having the following structure:

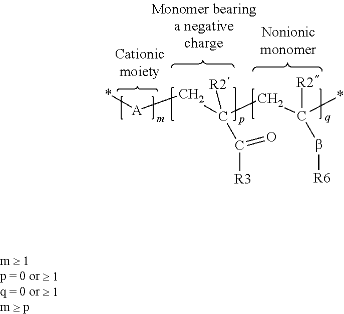

where A, may be one or more of the following cationic moieties:

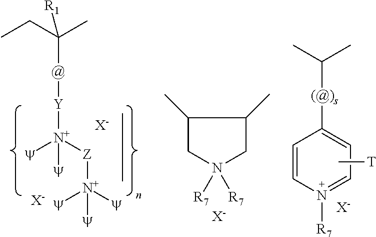

where:
@=amido, alkylamido, ester, ether, alkyl or alkylaryl;
Y=C1-C22 alkyl, alkoxy, alkylidene, alkyl or aryloxy;
ψ=C1-C22 alkyl, alkyloxy, alkyl aryl or alkyl arylox;
Z=C1-C22 alkyl, alkyloxy, aryl or aryloxy;
R1=H, C1-C4 linear or branched alkyl;
s=0 or 1, n=0 or ≥1;
T and R7=C1-C22 alkyl;
X⁻=halogen, hydroxide, alkoxide, sulfate or alkylsulfate;
the monomer bearing a negative charge is defined by R2′=H, C1-C4 linear or branched alkyl and R3 selected among:

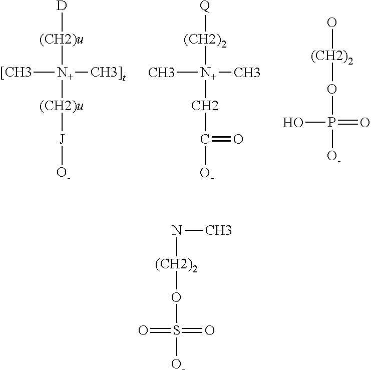

where D═O, N, or S;
Q=NH2 or O;
u=1, 2, 3, 4, 5 or 6;
t=0 or 1;
J=oxygenated functional group containing the following elements P, S, C;
And the nonionic monomer is defined by R2″=H, C1-C4 linear or branched alkyl, R6=linear or branched alkyl, alkyl aryl, aryl oxy, alkyloxy, alkylaryl oxy and β is defined as

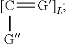

where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers include those which comprise a quaternary ammonium group of formula —N(Ra)₃⁺, wherein Ra, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chloride, bromide, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional suitable cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with a hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X⁻) in association with the synthetic cationic polymers may be any known counterion so long as the polymers remain soluble or dispersible in water, in the composition, or in a coacervate phase of the composition, and so long as the counterions are physically and chemically compatible with the essential components of the composition or do not otherwise unduly impair product performance, stability or aesthetics. Non limiting examples of such counterions include halides (e.g., chlorine, fluorine, bromine, iodine), sulfate and methylsulfate.

Cationic Cellulose Polymers (f)

Suitable cationic cellulose polymers are salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Non-limiting examples include: JR-400, JR-125, JR-30M, KG-30M, JP, LR-400 and mixtures thereof. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

Suitable cationic cellulose polymers may have a cationic charge density of from about 0.5 meq/gm to about 2.5 meq/gm, and/or from about 0.6 meq/gm to about 2.2 meq/gm, and/or from about 0.6 meq/gm to about 2.0 meq/gm. Further, the cationic charge density may be about 1.9 meq/gm. The polymers also have a molecular weight of from about 200,000 to about 3,000,000 g/mol, and/or from about 300,000 to about 2,200,000 g/mol, from about 1,000,000 to about 2,200,000 g/mol and/or from about 300,000 to about 1,500,000 g/mol. The cationic cellulose polymer may have a cationic charge density of about 1.7 to about 2.1 meq/gm and a molecular weight of from about 1,000,000 to about 2,000,000 g/mol.

In particular, the compositions according to the invention include at least 0.01% by weight, advantageously from 0.1% to 5% by weight, and preferably from 0.1% to 1% by weight of a cationic polymer B, and in particular of a cationic guar polymer, especially selected among those previously described, relative to the total weight of the composition.

According to preferred embodiments, the compositions according to the invention include guar hydroxypropyltrimonium chloride and/or hydroxypropyl guar hydroxypropyltrimonium chloride from the Solvay Jaguar® range, as cationic polymer B, and advantageously only guar hydroxypropyltrimonium chloride, hydroxypropyl guar hydroxypropyltrimonium chloride or a mixture thereof, as cationic polymer B.

Active Ingredient (C)

Any suitable active ingredients C, especially those which may be used in a personal care composition or in a household care composition can be included in the composition according to the invention. Such active ingredients are well known in the art, and for instance described in U.S. Pat. No. 10,058,498. The relevant part of U.S. Pat. No. 10,058,498 is included hereafter.

According to the invention, the compositions are preferably a personal care product or a household care product. A personal care product contains at least one active personal care ingredient C. Personal care compositions include hair care, skin care, sun care, nail care, and oral care compositions. An active personal care ingredient should provide some benefit to the user, when administered to the user, and in particular when applied to the skin or hair, in case of hair care or skin care products. The personal care active ingredients include, but are not limited to, anti-microbial agents, and in particular antibiotic agents, anti-fungal agents, antibacterial agents, antidandruff agents, antiseptic agents; analgesics, anesthetics, vitamins, hormones, antidiarrhea agents, corticosteroids, anti-inflammatory agents, vasodilators, kerolytic agents, dry-eye compositions, wound-healing agents, anti-infection agents, as well as solvents, diluents, adjuvants and other ingredients such as water, ethyl alcohol, isopropyl alcohol, propylene glycol, higher alcohols, glycerine, sorbitol, mineral oil, preservatives, surfactants, propellants, fragrances, essential oils, and viscosifying agents.

Other examples of active ingredients C that may suitably be included, but not limited to, in the personal care products corresponding of the compositions of the invention are as follows: 1) perfumes, which give rise to an olfactory response in the form of a fragrance and deodorant perfumes which in addition to providing a fragrance response can also reduce body malodor; 2) skin coolants, such as menthol, menthyl acetate, menthyl pyrrolidone carboxylate N-ethyl-p-menthane-3-carboxamide and other derivatives of menthol, which give rise to a tactile response in the form of a cooling sensation on the skin; 3) emollients, such as isopropylmyristate, silicone materials, mineral oils and vegetable oils which give rise to a tactile response in the form of an increase in skin lubricity; 4) deodorants other than perfumes, whose function is to reduce the level of or eliminate micro flora at the skin surface, especially those responsible for the development of body malodor. Precursors of deodorants other than perfume can also be used; 5) antiperspirant actives, whose function is to reduce or eliminate the appearance of perspiration at the skin surface; 6) moisturizing agents, that keep the skin moist by either adding moisture or preventing from evaporating from the skin; 7) cleansing agents, that remove dirt and oil from the skin; 8) sunscreen active ingredients that protect the skin and hair from UV and other harmful light rays from the sun; 9) hair treatment agents that condition hair, cleanse hair, detangle hair, act as styling agents, volumizing and gloss agents, color retention agents, antidandruff agents, hair growth promoters, hair dyes and pigments, hair perfumes, hair relaxer, hair bleaching agents, hair moisturizer, hair oil treatment agents, and antifrizzing agents; 10) oral care agents, such as dentifrices and mouth washes that clean, whiten, deodorize and protect the teeth and gum; 11) denture adhesives that provide adhesion properties to dentures; 12) shaving products such as creams, gels and lotions and razor blade lubricating strips; 13) tissue paper products such as moisturizing or cleansing tissues; 14) beauty aids such as foundation powders, lipsticks, and eye care; and 15) textile products such as moisturizing or cleansing wipes.

When the compositions of the invention are a household care product, this household care product includes at least one active household care ingredient C. The household care active ingredient should provide some benefit to the user. Examples of such active ingredients C that may suitably be included, but not limited to, according to the present invention are as follows: 1) perfumes, which give rise to an olfactory response in the form of a fragrance and deodorant perfumes which in addition to providing a fragrance response can also reduce odor; 2) insect repellent agent whose function is to keep insects from a particular area or attacking skin; 3) bubble generating agent such as surfactant that generates foam or lather; 4) pet deodorizer or insecticides such as pyrethrins that reduce pet odor; 5) pet shampoo agents and actives, whose function is to remove dirt, foreign material and germs from the skin and hair surfaces; 6) industrial grade bar, shower gel, and liquid soap actives that remove germs, dirt, grease and oil from skin, sanitizes skin, and conditions the skin; 7) all purpose cleaning agents that remove dirt, oil, grease, and germs from the surface in areas such as kitchens, bathroom, and public facilities; 8) disinfecting ingredients that kill or prevent growth of germs in a house or public facility; 9) rug and upholstery cleaning actives which lift and remove dirt and foreign particles from the surfaces and also deliver softening and perfumes; 10) a laundry softener active which reduces static and makes fabric feel softer; 11) laundry detergent ingredients which remove dirt, oil, grease, stains and kills germs; 12) laundry or detergent or fabric softener ingredients that reduce color loss during the wash, rinse, and drying cycle of fabric care; 13) dishwashing detergents which remove stains, food, germs; 14) toilet bowl cleaning agents which remove stains, kills germs, and deodorizes; 15) laundry prespotter actives which help in removing stains from clothes; 16) fabric sizing agents which enhance appearance of fabric; 17) vehicle cleaning actives which removes dirt, grease, etc., from vehicles and equipment; 18) lubricating agents which reduce friction between parts; and 19) textile products such as dusting or disinfecting wipes.

The above enumerated personal care and household care active ingredients are only examples and are not complete lists of active ingredients that can be used. Other ingredients that are used in these types of products are well known in the industry.

According to preferred embodiments, the compositions of the invention are hair care compositions, preferentially shampoo, and particularly antidandruff shampoo.

The active ingredients soluble in the surfactant-based system are preferred, including hydrophobic active ingredients, according to the invention.

According to preferred embodiments, the active ingredient C is as described in EP 0347199 or US 2020/0129402. In particular, as proposed in US 2020/0129402, the active ingredient is selected among anti-microbial and anti-fungal agents like octopirox, triclosan, climbazole, ciclopirox, rilopirox, MEA-Hydroxyoctyloxypyridinone, strobilurins, azoxystrobin, 1,10-phenanthroline, ketoconazole, benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. These active ingredients C are hydrophobic and particularly suitable for antidandruff shampoo.

The piroctone olamine, referred as octopirox (commercial name), is an active ingredient C particularly preferred according to the invention.

Advantageously, the active ingredient C is present in the composition in an amount of at least 0.01% by weight, advantageously from 0.05% to 10% by weight, and preferably from 0.1% to 5% by weight.

In particular, the compositions according to the invention include from 0.05 to 10% by weight, and preferably from 0.1% to 5% by weight of a hydrophobic active ingredient C, and in particular of an anti-microbial or an anti-fungal agent previously described, and advantageously from 0.05 to 5% by weight, and preferably from 0.1% to 1% by weight of piroctone olamine, as active ingredient C.

According to preferred embodiments, the compositions according to the invention include piroctone olamine, as active ingredient C, and advantageously only piroctone olamine, as active ingredient C.

Anionic Surfactant (D)

The compositions according to the invention also include an anionic surfactant D. The anionic surfactant D is different from the multi-tail surfactant A. The anionic surfactant D may be a multi-tail surfactant or it may not be a multi-tail surfactant. In any case, the anionic surfactant D is an additional ingredient, different from the multi-tail surfactant A. In the composition, the weight amount of this anionic surfactant D is higher than the weight amount of the multi-tail surfactant A. If several anionic surfactants D are present in the composition, their total quantity is taken into account. In the same way, if several multi-tail surfactants A are present in the composition, their total quantity is taken into account.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate (SLS), sodium laureth sulfate (SLES), ammonium lauryl sulfate (ALS) or ammonium laureth sulfate (ALES), preferably sodium lauryl sulfate (SLS) orsodium laureth sulfate (SLES).

The composition of the present invention can also include anionic surfactants selected from the group consisting of:
a) R1 O(CH2CHR3O)y SO3M;
b) CH3 (CH2)z CHR2 CH2 O (CH2 CHR3O)y SO3M; and
c) mixtures thereof,
where R1 represents CH3 (CH2)10, R2 represents H or a hydrocarbon radical comprising 1 to 4 carbon atoms such that the sum of the carbon atoms in z and R2 is 8, R3 is H or CH3, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols, F-T oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfate and sodium trideceth-3 sulfate. The composition of the present invention can also include sodium tridecyl sulfate.

The surfactant system can include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof.

The amino acid based anionic surfactant can be a glutamate, for instance an acyl glutamate. Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl Glutamate, sodium olivoyl glutamate, disodium olivoyl glutamate, sodium palmoyl glutamate, disodium palmoyl Glutamate, TEA-cocoyl glutamate, TEA-hydrogenated tallowoyl glutamate, TEA-lauroyl glutamate, and mixtures thereof.

The amino acid based anionic surfactant can be an alaninate, for instance an acyl alaninate. Non-limiting example of acyl alaninates can include sodium cocoyl alaninate, sodium lauroyl alaninate, sodium N-dodecanoyl-1-alaninate and combination thereof.

The amino acid based anionic surfactant can be a sulfosuccinate, anionic alkyl and alkyl ether sulfosuccinates and mixtures thereof.

Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

The amino acid based anionic surfactant can be a glycinate for instance an acyl glycinate. Non-limiting example of acyl glycinates can include sodium cocoyl glycinate, sodium lauroyl glycinate and combination thereof.

The composition can contain additional anionic surfactants selected from the group consisting of isethionates, sulfonates, sulfoacetates, glucose carboxylates, alkyl ether carboxylates, acyl taurates, and mixture thereof.

Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof.

Non-limiting examples of sulfonates can include alpha olefin sulfonates, linear alkylbenzene sulfonates, sodium laurylglucosides hydroxypropylsulfonate and combination thereof.

Non-limiting examples of sulfoacetates can include sodium lauryl sulfoacetate, ammonium lauryl sulfoacetate and combination thereof.

Non-limiting example of glucose carboxylates can include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof.

Non-limiting example of acyl taurates can include sodium methyl cocoyl taurate, sodium methyl lauroyl taurate, sodium methyl oleoyl taurate and combination thereof.

In particular, the compositions according to the invention include at least 5% by weight, advantageously from 5% to 20% by weight, and preferably from to 15% by weight of an anionic surfactant, and in particular of a sulfated anionic surfactant especially selected among those previously described, and advantageously from 5 to 20% by weight, and preferably from 8 to 15% by weight of sodium laureth sulfate.

According to preferred embodiments, the compositions according to the invention include a sulfated anionic surfactant, and in particular sodium laureth sulfate, as anionic surfactant D, and advantageously a sulfated anionic surfactant, and in particular only sodium laureth sulfate, as anionic surfactant D.

In one embodiment, the composition of the invention may be a sulfate-free composition. It means that the composition of the invention may be devoided of, i.e. may not contain any anionic surfactant which is a derivative of a sulfate (0 pbw).

The term “anionic surfactant which is a derivative of a sulfate” means surfactants comprising at least one anionic group or group that can be ionized into an anionic group, chosen from sulfate functions (—OSO3H or —OSO3-).

According to this specific embodiment, the following anionic surfactants are preferably not present in the composition according to the invention: salts of alkyl sulfates, of alkylamide sulfates, of alkyl ether sulfates, of alkylamido ether sulfates, of alkylaryl ether sulfates, of monoglyceride sulfates.

For instance, according to this specific embodiment, the following anionic surfactants are preferably not present in the composition according to the invention: sodium lauryl sulfate (SLS), sodium laureth sulfate (SLES), ammonium lauryl sulfate (ALS) or ammonium laureth sulfate (ALES).

Optional Other Surfactant(s)

In the composition of the invention, the surfactant system can be composed exclusively of the multi-tail surfactant and of the anionic surfactant, or may also include one or several other surfactant(s), also referred as co-surfactant. Non limiting examples of other cationic, zwitterionic, amphoteric, and non-ionic additional surfactants suitable for use in the compositions of the invention, and in particular in hair care compositions, are described in McCutcheon's, Emulsifiers and Detergents, 1989 Annual, published by M. C. Publishing Co., and U.S. Pat. Nos. 3,929,678, 2,658,072; 2,438,091; 2,528,378, US 2020/0129402 which are incorporated herein by reference in their entirety. As the optional additional surfactant is different from the multi-tail surfactant A, it does not include more than one tail chain, so, do not include for instance more than one hydrocarbon (i.e., alkyl) chain including at least 6 carbon atoms.

This(these) additional surfactant(s) or co-surfactant(s) may, in particular, be amphoteric, zwitterionic or non-ionic. Examples of such surfactants are more precisely described in U.S. Pat. No. 10,058,498, as described hereafter.

Nonionic surfactants can be broadly defined as compounds containing a hydrophobic moiety and a nonionic hydrophilic moiety. Examples of the hydrophobic moiety can be alkyl, alkyl aromatic, dialkyl siloxane, polyoxyalkylene, and fluoro-substituted alkyls. Examples of hydrophilic moieties are polyoxyalkylenes, phosphine oxides, sulfoxides, amine oxides, and amides. Nonionic surfactants marketed under the trade name SURFYNOL® (Air Products and Chemicals, Inc., Allentown, Pa.) are examples of such surfactants. Cationic surfactants may contain amino or quaternary ammonium hydrophilic moieties which are positively charged when dissolved in aqueous composition. Zwitterionic surfactants are exemplified by those which can be broadly described as derivatives of aliphatic quaternary ammonium, phosphonium, and sulfonium compounds, which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic water-solubilizing group, e.g., carboxy, sulfonate, sulfate, phosphate, or phosphonate. As they are different from the multi-tail surfactant A, they do not include more than one tail chain, so, do not include for instance more than one hydrocarbon (i.e., alkyl) chain including at least 6 carbon atoms. Examples of amphoteric surfactants which can be used in the compositions of the invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Sulfate-free surfactants can be broadly defined as single tail surfactants, that are generally free from salts or esters of sulfuric acid. Examples of sulfate-free surfactants include, but are not limited to, sodium lauroyl sarcosinate, sodium lauroamphoacetate, cocamidopropyl betain, and decyl glucoside.

Advantageously, when present, the additional surfactant, and in particular the amphoteric surfactant, as previously described, is present in the composition in an amount from 0.01 to 20% by weight, and preferably from 0.1% to 10% by weight. In particular, the compositions according to the invention include from 0.1% to 20% by weight, and preferably from 0.1% to 10% by weight of cocamidopropyl betain.

According to preferred embodiments, the compositions according to the invention include an amphoteric surfactant, and in particular cocamidopropyl betain, as additional surfactant D, and advantageously an amphoteric surfactant, and in particular only cocamidopropyl betain, as additional surfactant D.

Optional Oil

The compositions according to the invention may also comprise further component(s), in addition to those previously described. In particular, the compositions of the invention may comprise an oil, and in particular a non-toxic or cosmetic oil. Suitable cosmetic oils are, for instance, Cyclopentasiloxane, Cyclomethicone, Dimethicone, Dimethiconol, Amodimethicone, PEG/PPG Dimethicones, Cetyl Dimethicone, Stearyl Dimethicone, Stearoxy Dimethicone, Behenoxy Dimethicone, Polyisobutene, Petrolatum, Mineral Oil, Hydrogenated Polydodecene, Hydrogenated Polydecene, Polydecene, Isoamyl Cocoate, PPG-3 Myristyl Ether, PPG-11 Stearyl Ether, Dicaprylyl Ether, Dicaprylyl Carbonate, Cetearyl Isononanoate, Cetyl Ethylhexanoate, Diethyhexyl Carbonate, Cetyl Ricinoleate, Myristyl Myristate, Stearyl Heptanoate, Decyl Cocoate, Decyl Oleate, PPG-15 Stearyl Ether, Octyldodecanol, Isocetyl Palmitate, Cetearyl Ethylhexanoate, Ethylhexyl Palmitate, Ethylhexyl Stearate, Isopropyl Palmitate, PPG-14 Butyl Ether, Triisostearin, C12-15 Alkyl Benzoate, Phenoxyethyl Caprylate, Isopropyl Myristate, Caprylic/Capric Triglyceride, sunflower oil, olive oil, argan oil, mineral oil, castor oil, ricinus oil, cocoa oil, palm oil, coconut oil, avocado oil, almond oil, jojoba oil, corn oil, rapeseed oil, sesame oil, soybean oil, wheatgerm oil, walnut oil, Oleyl Erucate and mixtures thereof.

Advantageously, when present, an oil, and in particular a cosmetic oil, as previously described, represents not more than 10% of the weight of the composition So, the composition of the invention may include from 0 to 10% by weight, and preferably from 0 to 2% by weight, relative to the total weight of the composition, of an oil, and in particular a cosmetic oil, as previously described.

Solvent Carrier

The compositions according to the invention may also include a solvent carrier which is, typically, water or a mixture of water with another solvent.

Indeed, most of the compositions of the invention are in the form of pourable liquids (under ambient conditions). Such compositions will therefore typically comprise a solvent carrier, which, generally, is used to up to balance (i.e. to reach, with the other components, 100% of the total weight). It may typically represent at least 40% by weight, from about 40% to about 85% by weight, alternatively from about 45% to about 80% by weight, alternatively from about 50% to about 75% by weight of composition. Of course, the solvent used in the composition should be compatible with the other components of the disclosed compositions.

The carrier may comprise water, or a miscible mixture of water and organic solvent (s). The carrier of the compositions of the present invention may be water or water solutions of lower alkyl alcohols and/or polyhydric alcohols. The lower alkyl alcohols are, in particular, monohydric alcohols having 1 to 6 carbons, typically, ethanol and isopropanol. Polyhydric alcohols typically have from 3 to 6 carbon atoms and from 2 to 6 hydroxyl groups. Examples of polyhydric alcohols include propylene glycol, hexylene glycol, glycerin, and propane diol. Certain formulations of commercially available multi-tail surfactants include such solvent.

In particular, water may be used up to balance (i.e. to reach, with the other components, 100% of the total weight), and represents typically from 40 to 85% by weight of the composition.

Other Optional Ingredients

In the present invention, the composition may further comprise one or more additional optional ingredients. Suitable additional optional ingredients include, but are not limited to conditioning agents, silicone emulsions, gel networks, chelating agents, colorants, foam busters, anti-static agents, rheology modifiers and thickeners, suspension materials and structurants, pH adjusting agents and buffers, preservatives, pearlescent agents, anti-oxidants, viscosity-adjusting agents, opacifiers, and combinations thereof.

Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the compositions of the invention, in particular to personal care compositions.

In particular, the compositions according to the invention may comprise a viscosity modifier or hydrotope or solubility controller.

For more details on possible additional ingredients, reference can be made to US 2020/0129402.

In general, the total content of these optional ingredients, when one or several are present, does not exceed 10% by weight of the total weight of the composition.

Compositions and Uses of the Invention

The compositions of the invention can be prepared according to conventional techniques of mixture of components.

In one embodiment, the process to prepare a composition of the invention may include a pre-solubilisation step of the active ingredient C in the surfactant system made with the multi-tail surfactant A and the anionic surfactants D

In another embodiment, a composition of the invention may be prepared by mixing together the multi-tail surfactant A, the cationic polymer B, the active ingredient C, and the anionic surfactant D, without any pre-solubilisation step.

The preferred weight quantity given for the above mentioned disclosure are advantageously combined with each other's, preferably combining the preferred ranges according to their rank (in particular, the broadest ones together and the narrowest ones together).

So, for instance, according to some embodiments, the composition of the invention comprises:
a multi-tail surfactant A, which represents from 0.1 to 10% by weight, and preferably from 0.1 to 5% by weight, of the total weight of the composition,
a cationic polymer B, which represents from 0.1 to 5% by weight, and preferably from 0.1 to 1% by weight, of the total weight of the composition,
an active ingredient C, which represents from 0.05 to 10% by weight, and preferably from 0.1 to 5% by weight, of the total weight of the composition,
an anionic surfactant D which is different from the multi-tail surfactant A and which represents from 5 to 20% by weight, and preferably from 5 to 15% by weight, of the total weight of the composition,
   wherein, in the composition, the weight amount of the multi-tail surfactant A is lower than the weight amount of the anionic surfactant D.

Of course, the multi-tail surfactant A, the cationic polymer B, the active ingredient C, the anionic surfactant D, and the additional surfactant, the oil and the solvent when present, specifically described or preferred in the previous specification, are preferentially used.

So, for instance, according to some preferred embodiments, the composition of the invention comprises:
a dialkylsulfosuccinate, as multi-tail surfactant A, which represents less than 10% by weight and/or at least 0.5% by weight, preferentially from 1% to 7% by weight, and more preferentially from 1 to 5% by weight, of the total weight of the composition,
a cationic guar polymer, as the cationic polymer B, which represents at least 0.01% by weight, advantageously from 0.1 to 5% by weight, and preferably from 0.1 to 1% by weight, of the total weight of the composition,
a hydrophobic active ingredient C, and in particular of an anti-microbial or an anti-fungal agent, which represents from 0.05 to 10% by weight, and preferably from 0.1 to 5% by weight, of the total weight of the composition,
a sulfated anionic surfactant, as anionic surfactant D, which is different from the multi-tail surfactant A and which represents at least 5% by weight, advantageously from 5 to 20% by weight, and preferably from 5 to 15% by weight, of the total weight of the composition,
one or several additional surfactant(s) selected among sulfate-free single tail surfactants, which represents from 0.01 to 20% by weight, and preferably from 0.1 to 10% by weight, of the total weight of the composition,
wherein, in the composition, the weight amount of the multi-tail surfactant A is lower than the weight amount of the anionic surfactant D, and in particular, the weight amount of the dialkylsulfosuccinate is lower than the weight amount of the sulfated anionic surfactant.

For instance, according to some more preferred embodiments, the composition of the invention comprises:
dioctyl sodium sulfosuccinate, sodium bis(tridecyl)sulfosuccinate or a mixture thereof, as multi-tail surfactant A, which represents from 1 to 7% by weight, and preferably from 2 to 5% by weight, of the total weight of the composition,
a guar hydroxypropyltrimonium chloride, a hydroxypropyl guar hydroxypropyltrimonium chloride or a mixture thereof, as cationic polymer B, which represents from 0.1 to 5% by weight, and preferably from 0.1 to 1% by weight, of the total weight of the composition,
piroctone olamine, as active ingredient C, which represents from 0.05 to 5% by weight, and preferably from 0.1 to 1% by weight, of the total weight of the composition,
sodium laureth sulfate, as anionic surfactant D, which represents from 5 to 20% by weight, and preferably from 8 to 15% by weight, of the total weight of the composition, cocamidopropyl betain, as additional surfactant, which represents from 0.01 to 20% by weight, and preferably from 0.1 to 10% by weight, of the total weight of the composition,
wherein, in the composition, the weight amount of the multi-tail surfactant A is lower than the weight amount of the anionic surfactant D, and in particular, the weight amount of dioctyl sodium sulfosuccinate, sodium bis(tridecyl)sulfosuccinate or a mixture thereof, which is present in the composition, is lower than the weight amount of sodium laureth sulfate.

The compositions according to the invention may be liquid, gel or semi-solid compositions, foamed or foamable compositions. Preferably, the compositions of the invention are aqueous compositions.

As previously mentioned, the compositions of the invention are personal care compositions or household care compositions.

The compositions of the invention are intended to be deposited on a substrate (typically, skin, hair, scalp, textiles . . . ) where its action is needed. The active ingredient C on the targeted surface is better retained with the compositions of the invention, even after addition of water or after a rinsing step with water, as classically carried out with hair care compositions, like shampoo.

As used herein, personal care compositions includes products such as shampoos, shower gels, liquid hand cleansers, hair colorants, facial cleansers, and other surfactant-based liquid compositions.

According to preferred embodiments, the compositions of the invention are hair care compositions, preferentially shampoos, and particularly anti-dandruff shampoos.

For personal care applications, the total composition will be physiologically acceptable. So, any compounds that are not physiologically acceptable should be excluded from the composition or used in quantity which does not alter this property of the composition.

Preferentially, the compositions according to the invention, whatever their constituents, are different from the structured compositions as described in U.S. Pat. No. 9,320,697. “Structured composition” is to be understood as meaning a formulation which has a viscosity, which falls with increasing shear rate, in the shear rate range from 0.1 to 100 s⁻¹ and which has a yield point of ≥1 mPa. Both the viscosity and the yield point are measured using a rheometer, the measurement axis of which is stored in an air bearing. The viscosity drops in the stated shear rate range by 1-10 orders of magnitude, with 2-6 orders of magnitude being preferred. The measurements are carried out using a plate-plate geometry with a diameter of 40 mm at 25° C. The yield point is measured in oscillation at a frequency of 1 Hz at 25° C. with a plate-plate geometry with a diameter of 40 mm. The shear stress is varied from 0.001-100 Pa and the criterion taken for the yield point is the shear stress at which a deviation of 5% of the storage modulus from the plateau value of the linearly viscoelastic range is achieved.

According to the invention, the multi-tail surfactant A and in particular the combination multi-tail surfactant A/cationic polymer B is used for enhancing the deposition and/or retention of the active ingredient C, on the substrate where its action is needed. In the case of personal care compositions, the targeted substrate is the skin, nails, preferentially hair, or scalp. In the cases of household care composition, and in particular textile care composition, the targeted substrate is mainly a substrate to be cleaned, in particular, textiles. Most of all, when the compositions of the invention are intended to be rinsed with water, the multi-tail surfactant and in particular the combination multi-tail surfactant A/cationic polymer B is used to improved hold of the active ingredient on the targeted substrate when deposited. So, the quantity of active ingredient can be minimal, as it is well retained on the targeted substrate and so its action is boosted. This use is of course associated to the complete definition of the compositions according to the invention.

So, according to the invention, when the composition is a hair care composition (typically shampoo, and preferably antidandruff shampoo) or a textile care composition, its use may comprise the steps of:
(a) applying the composition on the targeted substrate; and
(b) before, during or after step(a), diluting the composition, with water such that the combination multi-tail surfactant A/cationic polymer B undergoes synergetic action for improving the deposition and/or retention of the active ingredient C on the targeted substrate.

The application may consist in a direct application or in a spreading of the composition of the present invention, on the substrate where it has to be delivered, in particular on the skin, keratinous tissue as the hair, scalp, or textiles.

The dilution means that the composition is soluble or dispersible or foamable in water.

Additionally, the compositions of the invention have, directly or after appropriate dilution for use, suitable viscosity properties and when they are a foam or foamable formulation, the combination multi-tail surfactant A/cationic polymer B helps to maintain the foamed form.

The examples below serve to illustrate the invention, but have no limiting character.

### EXAMPLES

Method to Assess the Efficiency of an Active Deposition Booster in Anti-Dandruff Shampoo

Preparation of Shampoo Formulations

The hair care composition is prepared by adding together the multi-tail surfactant A and the cationic polymer B to the rest of the suitable ingredients: surfactants, polymers, active ingredients, and the balance of water (i.e. to reach, with the other components, 100% of the total weight). The blend is stirred until homogenization. Optional ingredients such as fragrances, oils, dyes and pigments, viscosity modifiers, stabilizing agents, thickeners, pH adjusting agents, preservatives, pearlescers or opacifiers and natural hair nutrients can be also incorporated.

A process including a pre-mix of the active C in the multi-tail surfactant A is also considered effective.

The blend or a part of the blend can be heated up to 50-80° C. then cooled, to ensure a better homogenization, or an improved stability over time, or a higher efficiency of the boosters.

The targeted final viscosity and pH should be within respective ranges commonly considered to be acceptable for anti-dandruff shampoo composition.

Measurement of the Active Deposition

The composition is applied on the substrate—preferably hair—using a standardized protocol, including a rinsing step. Multi-applications can be considered if the natural intrinsic deposition of the active is very low.

The active is extracted by ethanol for a known duration. The concentration of the active in the solvent is then measured using HPLC method. Quantification is made by reference to a standard curve.

The efficiency of the active deposition booster is calculated as gram of active extracted per gram of substrate—preferably hair tress.

Non-Limiting Examples

The shampoo formulations were prepared following the same protocol as described previously, and applied on hair tresses as also described in the text above. The percentages of used ingredients represent the active part in weight of the ingredient. They are given in % by weight, relative to the total weight of the composition. The amount of active deposited on hair is measured by HPLC after extraction with ethanol. Each deposition of active presented in the following examples is calculated from the average of 3 replicated experiments.

The following examples are provided to facilitate an understanding of the present invention. The examples are not provided to limit the scope of the claims.

### Example 1

This example demonstrates the synergetic efficiency of both the multi-tail surfactant A (here a sulfosuccinate surfactant) and the cationic polymer B (guar hydoxypropyltrimonium chloride) in boosting the deposition of an anti-dandruff active C on hair (Piroctone Olamine).

The formulations of the studied compositions are listed in Table 1A and the obtained results are presented in Table 1B.

TABLE 1A

| | | Formulations | | | |
|---|---|---|---|---|---|
| Ingredients | | 1 | | | |
| | Role in | (control | | | |
| Chemical name | shampoo | A) | 2 | 3 | 4 |
| Sodium Laureth Sulfate | Anionic | 14.2 | 14.2 | 10 | 10 |
| (SLES) | Surfactant D | wt % | wt % | wt % | wt % |
| Sodium dioctyl | Multi-tail | — | — | 4.2 | 4.2 |
| sulfosuccinate | Surfactant A | | | wt % | wt % |
| Cocamidopropyl Betain | Co-Surfactant | 2 | 2 | 2 | 2 |
| (CAPB) | | wt % | wt % | wt % | wt % |
| Piroctone Olamine | Active | 0.5 | 0.5 | 0.5 | 0.5 |
| | ingredient C | wt % | wt % | wt % | wt % |
| Guar | Cationic | — | 0.5 | — | 0.5 |
| Hydroxypropyltrimonium | polymer | | wt % | | wt % |
| Chloride | B | | | | |

| Sodium chloride | Viscosity | up to 2% |
|---|---|---|
| | adjuster | |
| Preservatives, pH adjusters | | up to 1% |
| Water | Solvent | balance |

TABLE 1B

| | Formulations | | | |
|---|---|---|---|---|
| | 1 | | | |
| | (control | | | |
| | A) | 2 | 3 | 4 |
| PO deposit (ug of PO/g of hair) | 21 | 25.5 | 26.8 | 37.9 |
| Standard deviation | 1.0 | 1.2 | 1.8 | 2.5 |
| Active deposition improvement | — | 121% | 128% | 180% |
| versus control A | | | | |

Description of the ingredients listed in the Example 1:
RHODAPEX ESB 30HA1 (ID, Solvay, Sodium Laureth Sulfate
AEROSOL OT70PG, Solvay, Sodium Dioctyl Dulfosuccinate
MACKAM CAB 818, Solvay, Cocamidopropyl Betain
OCTOPIROX, Clariant, Piroctone Olamine
JAGUAR EXCEL®, Solvay, Guar Hydroxypropyltrimonium Chloride

### Examples 2-4

The deposition improvement of the composition 6, composition 8 or composition 7 compared to the control and the compositions 5 and 2 also shows a synergetic effect leading to a boosted deposition of active on hair.

The formulations of the studied compositions are listed in Table 2A and the obtained results are presented in Table 2B.

TABLE 2A

| Ingredients | | Formulations | | | | | |
|---|---|---|---|---|---|---|---|
| | Role in | 1 | | | | | |
| Chemical name | shampoo | (control A) | 5 | 2 | 6 | 7 | 8 |
| Sodium Laureth | Anionic | 14.2 wt % | 10 wt % | 14.2 wt % | 10 wt % | 10 wt % | 11.2 wt % |
| Sulfate (SLES) | Surfactant | | | | | | |
| | D | | | | | | |
| Sodium bis(tridecyl) | Multi-tail | — | 4.2 wt % | — | 4.2 wt % | 4.2 wt % | 3 wt % |
| sulfosuccinate | Surfactant | | | | | | |
| | A | | | | | | |
| Cocamidopropyl | Co- | 2 wt % | 2 wt % | 2 wt % | 2 wt % | 2 wt % | 2 wt % |
| Betain (CAPB) | Surfactant | | | | | | |
| Piroctone Olamine | Active | 0.5 wt % | 0.5 wt % | 0.5 wt % | 0.5 wt % | 0.5 wt % | 0.5 wt % |
| | ingredient | | | | | | |
| | C | | | | | | |
| Guar | Cationic | — | — | 0.5 wt % | 0.5 wt % | 0.2 wt % | 0.5 wt % |
| Hydroxypropyltrimonium | polymer B | | | | | | |
| Chloride | | | | | | | |

| Sodium chloride | Viscosity | up to 2% |
|---|---|---|
| | adjuster | |
| Preservatives, pH adjusters | | up to 1% |
| Water | Solvent | balance |

TABLE 2B

| | Formulations | | | | | |
|---|---|---|---|---|---|---|
| | 1 | | | | | |
| | (control A) | 5 | 2 | 6 | 7 | 8 |
| PO deposit (ug of | 21 | 21 | 25.5 | 158 | 60.9 | 155 |
| PO/g of hair) | | | | | | |
| Standard deviation | 1.0 | 1.3 | 1.2 | 2.1 | 2.8 | 9.1 |
| Active deposition | — | 100% | 121% | 752% | 290% | 738% |
| improvement versus | | | | | | |
| control A | | | | | | |

Description of the Ingredients Listed in these Formulations:
AEROSOL TR70E®, Solvay, Sodium bis(tridecyl) sulfosuccinate

The other ingredients are listed in the Example 1.

### Examples 5-6

In these examples, two concentrations of cationic polymer were tested and compared to the control without cationic polymer.

The formulations of the studied compositions are listed in Table 3A and the obtained results are presented in Table 1B.

TABLE 3A

| | | Formulations | | |
|---|---|---|---|---|
| Ingredients | | 9 | | |
| | Role in | (control | | |
| Chemical name | shampoo | B) | 10 | 11 |
| Sodium Laureth Sulfate | Anionic | 11.2 | | |
| (SLES) | Surfactant D | wt % | | |
| Sodium bis(tridecyl) | Multi-tail | 3 | | |
| sulfosuccinate | Surfactant A | wt % | | |
| Cocamidopropyl Betain | Co- | 2 | | |
| (CAPB) | Surfactant | wt % | | |
| Piroctone Olamine | Active | 0.5 | | |
| | ingredient C | wt % | | |
| Guar | Cationic | — | 0.2 | 0.5 |
| Hydroxypropyltrimonium | polymer B | | wt % | wt % |
| Chloride | | | | |

| Sodium chloride | Viscosity | up to 2% |
|---|---|---|
| | adjuster | |
| Preservatives, pH adjusters | | up to 1% |
| Water | Solvent | Balance |

TABLE 3B

Formulations

| | 9 | | |
|---|---|---|---|
| | (control B) | 10 | 11 |
| PO deposit (ug of PO/g of hair) | 22.2 | 103.5 | 172 |
| Standard deviation | | 2.6 | 3.8 |
| Active deposition improvement | — | 466% | 775% |
| versus control B | | | |

Description of the Ingredients Listed in the Example 3:
AEROSOL TR70HG®, Solvay, Sodium bis(tridecyl) sulfosuccinate

The Other Ingredients are Listed in the Example 1

### Examples 7-9

Several cationic polymers are tested in shampoo formulations and all lead to an enhanced deposition of anti-dandruff active.

The formulations of the studied compositions are listed in Table 4A and the obtained results are presented in Table 4B.

TABLE 4A

| | | Formulations | | | |
|---|---|---|---|---|---|
| Ingredients | | 9 | | | |
| | Role in | (control | | | |
| Chemical name | shampoo | B) | 10 | 12 | 13 |
| Variable | Cationic | None | 0.2 | 0.2 | 0.2 |
| | polymer | | wt % | wt % | wt % |
| | B | | JAGUAR | JAGUAR | JAGUAR |
| | | | Excel | C14S | C17 |

| Sodium Laureth | Anionic | 11.2 |
|---|---|---|
| Sulfate (SLES) | Surfactant | wt % |
| | D | |
| Sodium bis(tridecyl) | Multi-tail | 3 |
| sulfosuccinate | Surfactant | wt % |
| | A | |
| Cocamidopropyl | Co- | 2 |
| Betain (CAPB) | Surfactant | wt % |
| Piroctone Olamine | Active | 0.5 |
| | ingredient | wt % |
| | C | |
| Sodium chloride | Viscosity | up to 2% |
| | adjuster | |
| Preservatives, pH adjusters | | up to 1% |
| Water | Solvent | Balance |

TABLE 4B

Formulations

| | 9 | | | |
|---|---|---|---|---|
| | (control | | | |
| | B) | 10 | 12 | 13 |
| PO deposit (ug of PO/g of | 22.2 | 103.5 | 151.2 | 184.2 |
| hair) | | | | |
| Standard deviation | | 2.6 | 3.1 | 1.2 |
| Active deposition | — | 466% | 681% | 830% |
| improvement versus | | | | |
| control B | | | | |

Description of the Ingredients Listed in these Formulations:
JAGUAR Excel®, Solvay, Guar Hydroxypropyltrimonium Chloride
JAGUAR C14S®, Solvay, Guar Hydroxypropyltrimonium Chloride
JAGUAR C17®, Solvay, Guar Hydroxypropyltrimonium Chloride
AEROSOL TR7OHG®, Solvay, Sodium bis(tridecyl) sulfosuccinate

The Other Ingredients are the Same as in Examples 1-4.

### Examples 10-11

The following examples prove that at iso-antidandruff performance, with the addition of the multi-tail surfactant A and the cationic polymer B to the shampoo composition, the amount of active ingredient C in the formulation can be reduced.

The formulations of the studied compositions are listed in Table 5A and the obtained results are presented in Table 5B.

TABLE 5A

| | | Formulations | | |
|---|---|---|---|---|
| Ingredients | | 1 | | |
| | Role in | (control | | |
| Chemical name | shampoo | A) | 10 | 14 |
| Sodium Laureth Sulfate | Anionic | 14.2 | 11.2 | |
| (SLES) | Surfactant D | wt % | wt % | |
| Sodium bis(tridecyl) | Multi-tail | — | 3 | |
| sulfosuccinate | Surfactant A | | wt % | |
| Cocamidopropyl Betain | Co- | 2 | 2 | |
| (CAPB) | Surfactant | wt % | wt % | |
| Piroctone Olamine | Active | 0.5 | 0.5 | 0.3 |
| | ingredient C | wt % | wt % | wt % |
| Guar | Cationic | — | 0.2 | 0.5 |
| Hydroxypropyltrimonium | polymer B | | wt % | wt % |
| Chloride | | | | |

| Sodium chloride | Viscosity | up to 2% |
|---|---|---|
| | adjuster | |
| Preservatives, pH adjusters | | up to 1% |
| Water | Solvent | Balance |

TABLE 5B

Formulations

| | 1 | | |
|---|---|---|---|
| | (control A) | 10 | 14 |
| PO deposit (ug of PO/g of hair) | 21 | 103.5 | 101.5 |
| Standard deviation | 1.0 | 2.6 | 0.6 |
| Active deposition improvement | — | 493% | 483% |
| versus control A | | | |

Description of the Ingredients Listed in the Example 5
AEROSOL TR70HG®, Solvay, Sodium bis(tridecyl) sulfosuccinate

The Other Ingredients are Listed in the Example 1

1-25: (canceled)

26. A composition comprising:
a multi-tail surfactant A,
a cationic polymer B,
an active ingredient C,
an anionic surfactant D which is different from the multi-tail surfactant A,
wherein, in the composition, the weight amount of the multi-tail surfactant A is lower than the weight amount of the anionic surfactant D.

27. The composition according to claim 26, wherein the composition is a personal care composition or a household care composition.

28. The composition according to claim 26, wherein the multi-tail surfactant A comprises at least two linear or branched alkyl chains, with at least one alkyl chains comprising at least 6 carbon atoms.

29. The composition according to claim 26 comprising from 0.1% to 10% by weight of the multi-tail surfactant A, based on a total weight of the composition.

30. The composition according to claim 26, wherein the multi-tail surfactant A is a dialkylsulfosuccinate.

31. The composition according to claim 26, wherein the multi-tail surfactant A is dioctyl sodium sulfosuccinate, sodium bis(tridecyl)sulfosuccinate, or mixtures thereof.

32. The composition according to claim 26, wherein the cationic polymer B is a cationic guar polymer selected from guar hydroxypropyltrimonium chloride, hydroxypropyl guar hydroxypropyltrimonium chloride, and mixtures thereof.

33. The composition according to claim 26 comprising at least 0.01% by weight of the cationic polymer B, based on a total weight of the composition.

34. The composition according to claim 26 comprising at least 0.01% by weight of the active ingredient C, based on a total weight of the composition.

35. The composition according to claim 26 comprising from 0.05% to 10% by weight of the active ingredient C, based on a total weight of the composition, wherein the active ingredient C is selected from hydrophobic anti-microbial agents, hydrophobic anti-fungal agents, or mixtures thereof.

36. The composition according to claim 26 comprising from 0.05% to 5% by weight of the active ingredient C, based on a total weight of the composition, wherein the active ingredient C is piroctone olamine.

37. The composition according to claim 26 comprising at least 5% by weight of the anionic surfactant D, based on a total weight of the composition.

38. The composition according to claim 26, wherein the anionic surfactant D is a sulfated anionic surfactant.

39. The composition according to claim 26 comprising from 5% to 20% by weight of the anionic surfactant D, based on a total weight of the composition, wherein the anionic surfactant D is sodium laureth sulfate.

40. The composition according to claim 26, wherein the composition further comprises at least one additional surfactant selected from amphoteric, zwitterionic, and non-ionic surfactants.

41. The composition according to claim 26 further comprising an amphoteric surfactant, wherein the amphoteric surfactant is selected from sodium lauroyl sarcosinate, sodium lauroamphoacetate, cocamidopropyl betaine, decyl glucoside, and mixtures thereof.

42. The composition according to claim 26 further comprising from 0.01 to 20% by weight of an amphoteric surfactant, based on a total weight of the composition.

43. The composition according to claim 26 further comprising 10% by weight or less of an oil, based on a total weight of the composition.

44. The composition according to claim 26 further comprising from 40% to 85% by weight of water, based on a total weight of the composition.

45. The composition according to claim 26 further comprising a solvent selected from lower alkyl alcohols, polyhydric alcohols, and mixtures thereof.

46. The composition according to claim 45, wherein the solvent is selected from ethanol, isopropanol, propylene glycol, hexylene glycol, glycerin, propane diol, and mixtures thereof.

47. A method for improving the deposition, retention, or both of an active ingredient C to a substrate comprising applying to a targeted substrate a composition comprising a combination of a multi-tail surfactant A, a cationic polymer B, and the active ingredient C.

48. The method of claim 47, wherein the composition is a shampoo and the targeted substrate is hair, scalp, or both.

49. The method of claim 47, wherein the composition improves the retention of the active ingredient C after a rinsing step with water.
